(19) United States
(12) Patent Application Publication
(10) Pub. No.: US 2016/0262989 A1
(43) Pub. Date: Sep. 15, 2016

(54) COMPOSITION

(71) Applicant: Conopco, Inc., d/b/a UNILEVER, Englewood Cliffs, NJ (US)

(72) Inventor: Abid IFTIKHAR, Liverpool (GB)

(73) Assignee: Conopco, Inc., d/b/a UNILEVER, Englewood Cliffs, NJ (US)

(21) Appl. No.: 15/029,677

(22) PCT Filed: Oct. 16, 2014
(86) PCT No.: PCT/EP2014/072237

§ 371 Date: Apr. 15, 2016

(30) Foreign Application Priority Data
Oct. 22, 2013 (EP) 13189642.5

(51) Int. Cl. A61K 8/02; A61K 8/81; A61K 8/37; A61Q 5/02; A61K 8/04

(52) U.S. Cl. CPC A61K 8/0279; A61Q 5/02; A61K 8/046; A61K 8/37; A61K 8/8152; A61K 2800/31; A61K 2800/412

(57) ABSTRACT

Dry shampoo comprising a base and propellant, wherein the base comprises sebum absorber comprises hollow particles.

The present invention relates to a dry shampoo.

Dry shampoos are compositions for cleansing the hair without washing with water.

The product provides a suitable product to provide a clean feel and look on days when perhaps there is no opportunity, either through lack of time or lack of available water, to wash the hair.

Database GNPD [Online] Mintel November 2011 (2011-11) “Instant Dry Shampoo” XP002683579 Database accession no. 1669958 discloses a dry shampoo comprising aluminium starch octenylsuccinate, silica and glycerine. The composition also comprises butylenes glycol.

Database GNPD [Online] Mintel February 2011 (2012-02) “Instant Dry Shampoo” XP002683580 Database accession no. 1727707 discloses a dry shampoo comprising aluminium starch octenylsuccinate, silica and glycerine. The composition also comprises butylenes glycol.

Despite the prior art there remains a need for improved dry shampoos.

Accordingly, and in a first aspect, the present invention provides a dry shampoo comprising a base and propellant, wherein the base comprises a sebum absorber which comprises hollow particles.

We have surprisingly found that an improved dry shampoo can be formulated using hollow particles as sebum absorbers. The main benefit of using hollow particles is that a dry shampoo can be formulated without the need for ethanol as sebum solubiliser. Without the requirement for ethanol there is also removed the requirements for a lacquered aerosol can and also anti-caking agent since the traditional sebum absorbers cake in the presence of ethanol.

Preferably, the composition comprises substantially no alcohol. More preferably the composition comprises substantially no ethanol. More preferably, the composition comprises from 0 to 2% wt., most preferably 0 to 1% wt. of the composition alcohol. More preferably, the composition comprises from 0 to 2% wt., more preferably from 0 to 1% wt. of the composition ethanol.

Preferably, the base comprises from 5 to 20% wt. of the composition.

Preferably, the base comprises from 2 to 75% wt of the base sebum absorber.

Preferably, the sebum absorbers have an average particle size as measured using a Beckman Coulter (LS13320) of from 6 to 20 microns, more preferably from 7 to 11 microns.

Preferably, the sebum absorbers have an average oil absorption of from 1.0 to 5.0 ml/gm.

Preferred hollow sebum absorbers include crosslinked polymethyl methacrylates. Suitable commercially available examples include SUNPMMA C000130, SUNPMMA C000170 and SUNPMMA- P commercially available from Sunjin Chemical Co. Ltd.

Preferably, the dry shampoo comprises an oil which is preferably present at from 0.1 to 5% wt. of the base.

Preferably, the oil is isopropyl myristate, benzyl alcohol, PPG-3 benzyl ether myristate. The most preferred is isopropyl myristate.

Preferably, the dry shampoo comprises propylene glycol. Preferably any propylene glycol is present at from 0.5 to 5% wt. of the base.

In a composition according to the invention the propylene glycol reduces or prevents white residues being deposited on the hair. This is particularly problematic for users with dark hair.

The composition of the invention also comprises a propellant to suspend the base immediately before mixing and to facilitate its egress from its container.

The composition is stored in a pressurised container. Preferably, the container is an aerosol canister. More preferably, the container is an un-lacquered aerosol canister.

Suitable propellants are well known in the art and include butane, isobutene and propane.

In a second aspect the present invention provides the use of hollow sebum absorbers to clean the hair.

More preferably, the invention provides the use of hollow sebum absorbers in a dry shampoo to clean hair.

### EXAMPLE

The cleansing efficacy of different dry shampoo products comprising various different sebum absorbers. Two tests were carried out: sebum removal and the volume rig. These are two different ways of measuring cleansing efficacy.

The volume rig measures the reduction in volume on application of sebum to a virgin hair switch. Application of sebum results in significant volume reduction. Application of the dry shampoo results in an increase in hair switch volume as the sebum is removed.

The sebum removal test measures the amount of sebum remaining on the hair and the result is subtracted from 100.

| | Volume | Sebum |
|---|---|---|
| Test | Rig (mm²) | Remaining % |
| 1 | 88 | 0 |
| 2 | 118 | 0 |
| 3 | 101 | 0 |
| 4 | 81 | 20 |
| 5 | 37 | 100 |
| 6 | 100 | 0 |

1. SUNPMMA COCO170 ex. Sunjin Chemical Co. Ltd.
2. SUNPMMA COCO130 ex. Sunjin Chemical Co. Ltd.
3. SUNPMMA-P ex. Sunjin Chemical Co. Ltd.
4. Standard Dry shampoo product comprising modified starch as sebum absorber.
5. Sebum
6. Control (untreated clean switch)

### Formulations

| Order of | Raw | INCI | Amount |
|---|---|---|---|
| addition | materials | names | % w/w |
| 1 | Sebum Absorber | — | 16 |
| 2 | A-46 | Isobutane (85%), | 84 |
| | | Propane (15%) | |
| | | | 100.000 |

1. A substantially ethanol free dry shampoo composition comprising a base and propellant, wherein the base comprises sebum absorber comprising hollow particles, wherein the composition comprises from 0 to 2% ethanol.

2. Dry shampoo according to claim 1 wherein the base comprises from 5 to 20% wt. of the composition.

3. Dry shampoo according to claim 1 wherein the base comprises from 2 to 75% wt of the base sebum absorber.

4. Dry shampoo according to claim 1 wherein the sebum absorber comprises particles of average particle size from 6 to 25 micrometer.

5. Dry shampoo according to claim 1 wherein the hollow particles comprise crosslinked polymethyl (meth)acrylate.

6. Dry shampoo according to claim 1 further comprising an oil.

7. Dry shampoo according to claim 6, wherein the oil is present in an amount of from 0.1 to 5% wt of the total composition.

8. Dry shampoo according to claim 6 wherein the oil is isopropyl myristate.
